(19) United States
(12) Patent Application Publication
(10) Pub. No.: US 2011/0306650 A1
(43) Pub. Date: Dec. 15, 2011

(54) PROCESS FOR THE PREPARATION OF GLYCOPYRRONIUM CHLORIDE

(75) Inventors: Fausto PIVETTI, Parma (IT); Monica Bocchi, Parma (IT); Emanuele Ferrari, Parma (IT)

(73) Assignee: Chiesi Farmaceutici S.p.A., Parma (IT)

(21) Appl. No.: 13/159,872

(22) Filed: Jun. 14, 2011

(30) Foreign Application Priority Data
Jun. 14, 2010 (EP) 10165784.9

(51) Int. Cl. A61K 31/40; A61P 11/08; A61P 11/06; A61P 13/00; A61P 1/00; A61P 17/06; A61P 1/04; C07D 207/12; A61P 11/00

(52) U.S. Cl. 514/424; 548/556

(57) ABSTRACT

The invention concerns a method for preparing glycopyrronium chloride, and its use in pharmaceutical applications.

### CROSS REFERENCES TO RELATED APPLICATIONS

This application claims priority to European Patent Application No. 10165784.9, filed on Jun. 14, 2010, which is incorporated herein by reference in its entirety.

### BACKGROUND OF THE INVENTION

1. Field of the Invention

The present invention relates to processes for the preparation of glycopyrronium chloride. The synthesized product is suitable for use in pharmaceutical applications such as treatment of respiratory disease.

### DISCUSSION OF THE BACKGROUND

Glycopyrronium bromide is a muscarinic M3 anticholinergic agent used to reduce salivation associated with administration of certain anaesthetics, and as adjunctive therapy for peptic ulcers. It has also been reported to be effective in the treatment of asthmatic symptoms (Hansel et al., Chest, 2005; 128:1974-1979), which is incorporated herein by reference in its entirety.

Glycopyrronium bromide is commercially available and can be synthesized according to the process described in U.S. Pat. No. 2,956,062, which is incorporated herein by reference in its entirety.

Other counterions (including inter alia the chloride ion) have been mentioned as theoretical alternatives to the bromide counterion of glycopyrronium. WO 2006/100453, which is incorporated herein by reference in its entirety, proposes the use of the iodide, acetate and sulphate salts as an alternative to glycopyrronium bromide due to milling difficulties associated with the latter.

That same document discloses methods for preparing the alternative salts. In particular, it is suggested that glycopyrronium iodide can be prepared by a route analogous to that reported in U.S. Pat. No. 2,956,062 for the manufacture of glycopyrronium bromide, utilizing N-methylpyrrolidin-3-ol (NMP) and methyl hydroxycyclopentylmandelate (MCPM). An alternative proposal is to use glycopyrronium bromide as starting material for the manufacture of other glycopyrronium salts. For example, ion exchange techniques are alleged to be useful for exchange of bromide for iodide. Another suggested approach is to treat glycopyrronium bromide with silver sulfate or silver acetate to generate glycopyrronium sulfate or glycopyrronium acetate, respectively.

An important consideration for the synthesis of glycopyrronium salts is the desired composition and/or ratio of resulting stereoisomers. Glycopyrrolate has two chiral centres corresponding to four isomeric forms comprising 2 pairs of diastereisomers, namely (3S,2′R)-, (3R,2′S)-, (3R,2′R)-, and (3S,2′S)- [(cyclopentyl-hydroxyphenylacetyl)oxy]-1,1-dimethylpyrrolidinium bromide. Commercially available glycopyrronium bromide consists of the purified “threo” diastereisomer (3R,2′S+3S,2′R). Differing pharmacological properties have been attributed to each of the individual isomers of glycopyrronium bromide.

Thus, it would be desirable to be able to synthesize pharmaceutical grade glycopyrronium chloride of suitable isomeric composition by means of a validated method that can ideally be carried out economically on a large scale.

### SUMMARY OF THE INVENTION

Accordingly, it is one object of the present invention to provide novel methods for synthesizing glycopyrronium chloride.

It is another object of the present invention to provide novel methods for the preparation of diastereoisomerically pure glycopyrronium chloride.

It is another object of the present invention to provide novel glycopyrronium chloride prepared by such a method.

It is another object of the present invention to provide novel pharmaceutical compositions which contain such glycopyrronium chloride.

It is another object of the present invention to provide novel methods of preventing and/or treating certain diseases and conditions by administering an effective amount of such glycopyrronium chloride.

These and other objects, which will become apparent during the following detailed description, have been achieved by the inventors' discovery of methods for preparing glycopyrronium chloride.

Thus, in a first aspect the present invention provides methods for synthesizing glycopyrronium chloride from glycopyrronium acetate, comprising the step of reacting the glycopyrronium acetate with hydrogen chloride to generate glycopyrronium chloride. Preferably, the glycopyrronium acetate is first prepared from glycopyrronium bromide by a step comprising reacting the glycopyrronium bromide with silver acetate to generate said glycopyrronium acetate.

In a second aspect the present invention provides methods for synthesizing glycopyrronium chloride from glycopyrronium bromide characterized by contacting the glycopyrronium bromide with an ion exchange resin, wherein the resin is preferably preconditioned with sodium chloride.

In a third aspect, the present invention provides methods for synthesizing glycopyrronium chloride from 3-[(cyclopentylhydroxyphenylacetyl)oxy]-1-methylpyrrolidine by treatment with methane chloride, optionally followed by one or more successive recrystallizations.

In a fourth aspect, the present invention provides methods for preparation of diastereoisomerically pure glycopyrronium chloride comprising dissolving glycopyrronium chloride in hot acetonitrile and then cooling the solution to allow crystallization of diastereoisomerically pure glycopyrronium chloride.

In yet another aspect, the present invention provides glycopyrronium chloride prepared by the methods of the invention.

In a further aspect, the present invention provides diastereoisomerically pure glycopyrronium chloride, preferably having a (R,R)+(S,S) diastereoisomer content of less than 20% w/w.

In yet another aspect, the present invention provides pharmaceutical compositions comprising diastereoisomerically pure glycopyrronium chloride, and/or glycopyrronium chloride prepared according to a method of the invention, and one or more pharmaceutically acceptable excipients.

In a further aspect the present invention provides diastereoisomerically pure glycopyrronium chloride for prevention or treatment of any of: COPD (chronic bronchitis and emphysema); asthma; acute lung injury (ALI); cystic fibrosis; rhinitis; adult or respiratory distress syndrome (ARDS); urinary incontinence; irritable bowel syndrome; psoriasis; hyperhydrosis; sialorrhea; and gastrointestinal ulcers.

In yet another aspect, the present invention provides methods for the prevention or treatment of any of: COPD (chronic bronchitis and emphysema); asthma; acute lung injury (ALI); cystic fibrosis; rhinitis; adult or respiratory distress syndrome (ARDS); urinary incontinence; irritable bowel syndrome; psoriasis; hyperhydrosis; sialorrhea; and gastrointestinal ulcers by administering an effective amount of diastereoisomerically pure glycopyrronium chloride to a subject in need thereof.

### DETAILED DESCRIPTION OF THE PREFERRED EMBODIMENTS

The present inventors have observed that glycopyrronium chloride has several advantages over glycopyrronium bromide with respect to pharmaceutical formulations. In particular, glycopyrronium chloride is more soluble in ethanol and HFA134a/ethanol mixtures than glycopyrronium bromide, and it has also been found to have better compatibility with other active ingredients, especially with formoterol.

The first synthesis method of the invention (Method 1) comprises the synthesis from glycopyrronium bromide via glycopyrronium acetate as an intermediate. In a first step, glycopyrronium bromide is reacted with silver acetate to create glycopyrronium acetate. Preferably, this step is carried out in the presence of methanol, in which the silver acetate is dissolved: silver bromide precipitates from the reaction mixture and can be removed by filtration. Alternatively, glycopyrronium acetate can be prepared by any known method, such as that described in WO2006/100453, which is incorporated herein by reference in its entirety.

In the second step, glycopyrronium acetate, which is preferably dissolved in ethyl acetate, is reacted with hydrogen chloride: and glycopyrronium chloride crystallizes from the ethyl acetate solution.

In a subsequent step, crude glycopyrronium chloride can be purified by any conventional means, such as by crystallization or suspension.

In a preferred purification step applicable to glycopyrronium chloride prepared according to any of the three methods of the present invention, glycopyrronium chloride is dissolved in acetonitrile (for instance hot acetonitrile, e.g. at a temperature of 50 to 82° C. and then crystallized by cooling (for instance at a temperature of 0 to 20° C.). Reiteration of this recrystallization process leads to an increasingly diastereoisomerically pure final product having a desirably low content of (R,R)+(S,S) diastereoisomers.

Method 1 is ideally suited to small-scale synthesis.

The second synthesis method (Method 2) is adaptable for larger-scale synthesis. This method relies on the application of ion exchange technology. A column of anion exchange resin is prepared and activated by treatment with, for example, a NaCl solution, then loaded with glycopyrronium bromide. The anion exchange occurs on the column when glycopyrronium bromide is allowed to flow through the column: bromide ions are withdrawn by the resin and exchanged with chloride ions as counterions of glycopyrronium. Glycopyrronium chloride is then eluted from the column with an appropriate solvent or solvent mixture, such as ethanol or an ethanol/water mixture.

Suitable ion exchange resins are commercially available and include strong anion exchange resins like Amberlite® IRA900 or FAP90. The amount of resin should be adjusted on the basis of the amount of glycopyrronium bromide to be loaded and of the exchange capacity of the resin itself, as number of chloride equivalents per kg or liter of resin. Suitable excesses of resin chloride equivalents, generally 2 to 5 equivalents versus bromide equivalents to be loaded, are generally considered appropriate in order to get low bromide residue.

Resins are preferably loaded in glass columns of suitable diameter and length. If not already activated as chloride anion exchange, resins can be activated by contacting with an aqueous solution of sodium chloride, generally 5 to 10% p/v; elution with water follows to remove excess sodium chloride and finally the column is conditioned with the solvent to be used in glycopyrronium elution.

Glycopyrronium bromide is dissolved in appropriate volumes of a suitable solvent and the solution is loaded at the top of the resin column. Then eluting solvent is applied to the column: elution can occur by gravitation or through the use of a pump: in case of gravitation, flow is regulated through the height of the solvent reservoir, in case of pumping, flow is regulated by the pump speed. Solvent flow rate should be regulated on the basis of the bed volume in order to allow sufficient residence time of glycopyrronium within the column.

Glycopyrronium chloride solution is collected at the exit of the column: several fractions are collected of suitable volume, depending on the column bed volume. After analytical checks (e.g. by TLC), suitable fractions are blended for the following work-up and isolation.

The pooled fractions may be decolored (e.g. with charcoal). They can be filtered, for instance through mineral filters such as Dicalite®. The pooled fractions can be concentrated by evaporation, for example through use of a rotary evaporator. For optimum purity the residue obtained after concentration can be re-suspended in ethyl acetate and concentrated again in order to remove water as an azeotrope.

Optional further purification can be carried out as described earlier by dissolution in hot acetonitrile and crystallization by cooling.

The third synthesis method (Method 3) comprises a step analogous to that disclosed in U.S. Pat. No. 2,956,062, which is incorporated herein by reference in its entirety. 3-[(cyclopentylhydroxyphenylacetyl)oxy]-1-methylpyrrolidine as a mixture of (R,R),(R,S),(S,S),(S,R) isomers, preferably dissolved in acetone, is first reacted with methyl chloride. However, methyl chloride has very different chemical properties from the methyl bromide used in the method of U.S. Pat. No. 2,956,062. In particular, methyl chloride has a boiling point of −24.2° C., compared to +4° C. for methyl bromide. It is known that by reacting 3-[(cyclopentylhydroxyphenylacetyl)oxy]-1-methylpyrrolidine with methyl bromide in toluene and/or acetone according to U.S. Pat. No. 2,956,062 a product is obtained with a diastereoisomeric profile of 60% threo, 40% erythro. The potential diastereoisomeric profile of the intermediate product obtained by treating 3-[(cyclopentylhydroxyphenylacetyl)oxy]-1-methylpyrrolidine with methyl chloride could not have been predicted prior to attempting the present synthesis.

The subsequent recrystallization of glycopyrronium chloride in solvent mirrors the equivalent step in U.S. Pat. No. 2,956,062. However, prior to actually carrying out the present inventive Method 3, the skilled person could not have foreseen whether the diastereoselectivity of the chloride end product following recrystallization would be identical to that of the bromide.

In an alternative embodiment of method 3 (method 4) in step (a) 3-[(cyclopentyl-hydroxyphenylacetyl)oxy]-1-methylpyrrolidine in the form of a mixture of (R,S), (S,R), (S,S), (R,R) isomers is first treated with an appropriate acid in order to crystallize the desired (R,S), (S,R) diastereoisomer as a suitable salt. Diastereoisomeric purity can be enhanced in step (b) by recrystallization of the (R,S),(S,R)-3-[(cyclopentyl-hydroxyphenylacetyl)oxy]-1-methylpyrrolidine salt in a suitable solvent or solvent mixture. Finally, in step (c) diasteroisomerically pure (R,S),(S,R)-3-[(cyclopentyl-hydroxyphenylacetyl)oxy]-1-methylpyrrolidine free base is generated by alkaline treatment of the salt obtained in step (b) and extraction in organic solvent. Then, in step (d), the free base is converted to glycopyrronium chloride by reaction with methyl chloride through conventional methods, using toluene and/or acetone as described above. In step (a), the appropriate acid to isolate the desired (R,S), (S,R) diastereoisomer of 3-[(cyclopentylhydroxyphenylacetyl)oxy]-1-methylpyrrolidine may be selected from the group of benzoic acid, 3-chlorobenzoic acid, 3-nitrobenzoic acid, isophtalic acid, 5-nitroisophtalic acid, phosphoric acid, methanesulfonic acid, benzenesulfonic acid, fumaric acid, and maleic acid, and the reaction is operated at a temperature in the range from 0 to 40° C. and preferably from 10 to 30° C.

In step (b), suitable solvents for the crystallization of the desired (R,S), (S,R) salt may be selected from the group constituted by methanol, ethanol, isopropanol, methyl-ethylketone, ethyl acetate, water, and acetonitrile. The mixture is heated at a temperature from 20 up to 80° C. and then cooled at a temperature from 0 to 20° C. to crystallize the desired salt.

In step (c), the alkaline treatment of the salt of the desired diastereoisomer may be carried out by treatment with a base selected from the group of sodium hydroxide, sodium hydrogencarbonate, sodium carbonate and potassium carbonate. The extraction of the free base may be operated by using an organic solvent which may be selected from the group of toluene, ethyl acetate, isopropylacetate, and methyl t-butyl ether.

An optional purification step can be performed with hot acetonitrile followed by crystallization by cooling, as described above.

Preferably, diastereoisomerically pure glycopyrronium chloride prepared according to each of the methods of the invention can be defined as having a (R,R)+(S,S) diastereoisomer content of less than 40% w/w, more preferably less than 30% w/w, more preferably less than 20% w/w, more preferably less than 10% w/w, more preferably less than 5% w/w, more preferably less than 1% w/w, and most preferably less than 0.1% w/w.

The diastereoisomeric purity of glycopyrronium chloride can be determined by methods familiar to those skilled in the art, such as HPLC, GC, and NMR spectroscopy.

Pharmaceutical compositions can be prepared by admixture of glycopyrronium chloride prepared according to the present invention and one or more pharmaceutically acceptable excipients. Depending on the nature of the medical disease or condition to be treated, and the type of patient, the pharmaceutical compositions may be formulated to be delivered by any suitable route, including oral, intravenous, parenteral, inhalation, intranasal, topical, subcutaneous, intramuscular, rectal, or vaginal. Suitable dosage forms include all those known to the skilled person, such as tablets, capsules, powders, sustained release formulations, ointments, gels, creams, suppositories, eye drops, transdermal patches, syrups, solutions, suspensions, aerosols, solutions for nebulizers, nasal sprays, etc. In a preferred embodiment, the composition is formulated for delivery by the inhalation or intranasal routes, for instance in an aerosol solution or suspension, as a dry powder for inhalation, or in a nasal spray.

Suitable excipients include carriers, diluents, wetting agents, emulsifying agents, binders, coatings, fillers, glidants, lubricants, disintegrants, preservatives, surfactants, pH buffering substances and the like. Examples of excipients and their use are provided in the Handbook of Pharmaceutical Excipients, 5^{th} ed. (2006), Ed. Rowe et al., Pharmaceutical Press., which is incorporated herein by reference in its entirety.

Suitable dosages of glycopyrronium chloride in the pharmaceutical compositions of the present invention may easily be established by the attending physician and will depend on the type of patient and nature of the disease or condition, and on the mode of drug delivery. Dosage levels of the order of about 0.1 μg to about 25 mg per kilogram of body weight per day may be useful. For prevention or treatment of respiratory conditions glycopyrronium chloride is likely to be delivered by inhalation, in which case the preferred dosage is probably about 0.5 to 100 μg per inhalation device actuation, preferably about 1 to 40 μg per actuation, and more preferably about 5 to 26 μg per actuation.

The glycopyrronium chloride obtained according to the present invention may be used for prophylactic purposes or for symptomatic relief for a wide range of conditions including: respiratory disorders such as chronic obstructive pulmonary disease (COPD) and asthma of all types. Other respiratory disorders for which the product of the invention may be beneficial are those characterized by obstruction of the peripheral airways as a result of inflammation and presence of mucus, such as chronic obstructive bronchiolitis, chronic bronchitis, emphysema, acute lung injury (ALI), cystic fibrosis, rhinitis, and adult or respiratory distress syndrome (ARDS).

In addition, glycopyrronium chloride synthesized according the present invention may be useful in treating smooth muscle disorders such as urinary incontinence and irritable bowel syndrome; skin diseases such as psoriasis; hyperhydrosis and sialorrhea; and gastrointestinal ulcers.

In one embodiment of the present invention there is provided use of diastereoisomerically pure glycopyrronium chloride and/or glycopyrronium chloride prepared according to any of the methods of the invention, in the manufacture of a medicament for the prevention or treatment of any of: COPD (chronic bronchitis and emphysema); asthma; acute lung injury (ALI); cystic fibrosis; rhinitis; adult or respiratory distress syndrome (ARDS); urinary incontinence; irritable bowel syndrome; psoriasis; hyperhydrosis; sialorrhea; and gastrointestinal ulcers.

In a further embodiment of invention there is provided a method for prevention of treatment of any of: COPD (chronic bronchitis and emphysema); asthma; acute lung injury (ALI); cystic fibrosis; rhinitis; adult or respiratory distress syndrome (ARDS); urinary incontinence; irritable bowel syndrome; psoriasis; hyperhydrosis; sialorrhea; and gastrointestinal ulcers in a patient, comprising the administration to the patient of a therapeutically effective amount of diastereoisomerically pure glycopyrronium chloride and/or glycopyrronium chloride prepared according to any of the methods of the invention. A “therapeutically effective amount” of substance is defined herein as an amount leading to a detectable improvement in one or more clinical symptoms of the treated condition or measurably reducing the probability of development of a disease condition or its symptoms.

Other features of the invention will become apparent in the course of the following descriptions of exemplary embodiments which are given for illustration of the invention and are not intended to be limiting thereof

### EXAMPLES

### Example 1

### Preparation of Glycopyrronium Chloride According to Method 1.

Glycopyrronium bromide (25.0 g, 0.063 mol) was dissolved in methanol (750 ml). Silver acetate (10.5 g, 0.063 mol) was added, and the mixture was stirred for 2 hours at 15 to 25° C.: precipitation of silver bromide occurred. The solid was filtered through a Dicalite® pad, and the filtered solution was concentrated in a rotary evaporator. Residual oily glycopyrronium acetate was dissolved in ethyl acetate (150 ml) and a 4.2M solution of hydrogen chloride in ethyl acetate (18 ml, 0.076 mol) was added dropwise causing crystallization of glycopyrronium chloride. The suspension was stirred for 1 hour at 5 to 10° C., then it was filtered and the solid was dried.

Crude glycopyrronium chloride (18.6 g, 0.053 mol) was dissolved in hot acetonitrile and crystallized by cooling at 5 to 10° C. for 2 hours. After filtering and drying at 50° C. under vacuum for 16 hours, glycopyrronium chloride (16.0 g, 0.045 mol) was recovered as a white powder with 72% yield.

### Example 2

### Preparation of Glycopyrronium Chloride According to Method 2

Resin Amberlite® IRA900 Cl (500 g) was suspended in 1500 ml of a mixture of ethanol/water 50/50 v/v and loaded in a glass column of 60 mm internal diameter with bottom filter and valve. The excess solvent was allowed to pass through the column: the bed height was about 25 cm, corresponding to a bed volume of 700 ml. Glycopyrronium bromide (74 g, 0.186 mol) was dissolved in 280 ml of a mixture of ethanol/water 50/50 v/v and loaded at the top of the column. The solution was passed through the column followed by a mixture of ethanol/water 50/50 v/v as eluting solvent. Elution occurred by gravitation and the flow rate was adjusted to 15 to 20 ml/min; 80 to 100 ml fractions were collected at the bottom of the column and analyzed for glycopyrronium content (by TLC as from pharmacopeia): glycopyrronium started eluting in fraction 3, its concentration was at a maximum in fractions 5 to 8 and then decreased until it disappeared in fraction 17. Fractions 3 to 16 were blended, and the resulting solution (1.41) was decolored with charcoal, filtered through a Dicalite® layer and concentrated in a rotary evaporator. The oily residue was suspended in ethyl acetate (740 ml) and concentrated again in order to remove water as azeotrope; after partial concentration and addition of fresh ethyl acetate, glycopyrronium chloride crystallized out as a white powder. The suspension was stirred and cooled at 0° C. and the solid was filtered and dried under vacuum at 50° C. Glycopyrronium chloride (65.0 g, 0.175 mol) was obtained as the monohydrate crystal, with 94% yield.

The obtained product was characterized by having more than 99% purity, 100.1% assay, less than 0.1% (R,R)(S,S) diasteroisomer, 9.9% chloride content, 138 ppm bromide content.

### Example 3

### Preparation of Glycopyrronium Chloride According to Method 3

3-[(cyclopentylhydroxyphenylacetyl)oxy]-1-methylpyrrolidine as a mixture of (R,R),(R,S),(S,S),(S,R) isomers (20 g) was dissolved in acetone (80 ml). Methyl chloride (2,5 equivalents) was slowly bubbled into the solution over 6 hours while cooling the solution at 5° C. After 4 hours the product started precipitating. The flask was closed and stirred overnight at room temperature. The crystallized white powder was filtered and dried under vacuum: glycopyrronium chloride was isolated with 63% yield as a 58/42 (R,S),(S,R)/(R,R),(S,S) diastereoisomeric mixture.

The solid was suspended in hot acetonitrile (10 vol) and crystallized by cooling, leading to a product with 57% yield and 80/20 (R,S),(S,R)/(R,R),(S,S) diastereoisomeric ratio. Repeating the crystallization procedure from acetonitrile led to glycopyrronium chloride with 71% yield and 90/10 (R,S),(S,R)/(R,R),(S,S) diastereoisomeric ratio. Generation of product of even greater diastereoisomeric purity was possible by repeating crystallizations from acetonitrile.

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A method for the preparation of glycopyrronium chloride from glycopyrronium acetate, comprising:
reacting glycopyrronium acetate with hydrogen chloride to obtain glycopyrronium chloride.

2. A method according to claim 1, wherein said glycopyrronium acetate is prepared from glycopyrronium bromide by reacting the glycopyrronium bromide with silver acetate to obtain said glycopyrronium acetate.

3. A method according to claim 2, wherein said reacting said glycopyrronium bromide with silver acetate is performed in methanol.

4. A method according to claim 1, wherein said glycopyrronium acetate is dissolved in ethyl acetate before addition of said hydrogen chloride to obtain glycopyrronium chloride.

5. A method according to claim 2, wherein said glycopyrronium acetate is dissolved in ethyl acetate before addition of said hydrogen chloride to obtain glycopyrronium chloride.

6. A method according to claim 3, wherein said glycopyrronium acetate is dissolved in ethyl acetate before addition of said hydrogen chloride to obtain glycopyrronium chloride.

7. A method for the preparation of glycopyrronium chloride from glycopyrronium bromide, comprising:
contacting glycopyrronium bromide with an ion exchange resin.

8. A method according to claim 7, wherein said ion exchange resin is preconditioned with sodium chloride.

9. A method according to claim 7, wherein said glycopyrronium chloride is eluted from said ion exchange resin with ethanol or an ethanol/water mixture.

10. A method according to claim 8, wherein said glycopyrronium chloride is eluted from said ion exchange resin with ethanol or an ethanol/water mixture.

11. A method for the preparation of glycopyrronium chloride, comprising:
(a) treating 3-[(cyclopentylhydroxyphenylacetyl)oxy]-1-methylpyrrolidine in the form of a mixture of (R,S), (S,R), (S,S), (R,R) isomers with at least one acid to crystallize the (R,S), (S,R) diastereoisomer as a salt;
(b) recrystallizing said (R,S),(S,R)-3-[(cyclopentylhydroxyphenylacetyl)oxy]-1-methylpyrrolidine salt from a solvent or solvent mixture, to obtain a recrystallized salt;
(c) treating said recrystallized salt with at least one base to obtain diasteroisomerically pure (R,S),(S,R)-3-[(cyclopentyl-hydroxyphenyl acetyl)oxy]-1-methylpyrrolidine free base; and
(d) converting said free base to glycopyrronium chloride by reaction with methyl chloride.

12. A method according to claim 11, wherein said 3-[(cyclopentyl-hydroxyphenylacetyl)oxy]-1-methylpyrrolidine in the form of a mixture of (R,S), (S,R), (S,S), (R,R) isomers is treated with at least one acid selected from the group consisting of benzoic acid, 3-chlorobenzoic acid, 3-nitrobenzoic acid, isophtalic acid, 5-nitroisophtalic acid, phosphoric acid, methanesulfonic acid, benzenesulfonic acid, fumaric acid, and maleic acid.

13. A method according to claim 11, wherein said 3-[(cyclopentyl-hydroxyphenylacetyl)oxy]-1-methylpyrrolidine in the form of a mixture of (R,S), (S,R), (S,S), (R,R) isomers is treated with said at least one acid at a temperature in the range of 0 to 40°.

14. A method according to claim 11, wherein said (R,S),(S,R)-3-[(cyclopentylhydroxyphenylacetyl)oxy]-1-methylpyrrolidine salt is recrystallized from a solvent or solvent mixture selected from the group consisting of methanol, ethanol, isopropanol, methyl-ethylketone, ethyl acetate, water, and acetonitrile.

15. A method according to claim 11, wherein said recrystallizing said (R,S),(S,R)-3-[(cyclopentylhydroxyphenylacetyl)oxy]-1-methylpyrrolidine salt from a solvent or solvent mixture comprises heating a crystallization mixture to a temperature of 20 to 80° C. and then cooling said crystallization mixture to a temperature of 0 to 20° C.

16. A method for the preparation of diastereoisomerically pure glycopyrronium chloride, comprising:
dissolving glycopyrronium chloride in hot acetonitrile, to obtain a solution, and then cooling said solution to allow crystallization of diastereoisomerically pure glycopyrronium chloride.

17. Glycopyrronium chloride prepared by the method according to claim 1.

18. Glycopyrronium chloride prepared by the method according to claim 7.

19. Glycopyrronium chloride prepared by the method according to claim 11.

20. Diastereoisomerically pure glycopyrronium chloride.

21. Diastereoisomerically pure glycopyrronium chloride according to claim 20, having a (R,R)+(S,S) diastereoisomer content of less than 10% w/w.

22. A pharmaceutical composition, comprising diastereoisomerically pure glycopyrronium chloride and one or more pharmaceutically acceptable excipients.

23. A pharmaceutical composition, comprising glycopyrronium chloride prepared by a method according to claim 1 and one or more pharmaceutically acceptable excipients.

24. A pharmaceutical composition, comprising glycopyrronium chloride prepared by a method according to claim 7 and one or more pharmaceutically acceptable excipients.

25. A pharmaceutical composition, comprising glycopyrronium chloride prepared by a method according to claim 11 and one or more pharmaceutically acceptable excipients.

26. A pharmaceutical composition, comprising glycopyrronium chloride prepared by a method according to claim 16 and one or more pharmaceutically acceptable excipients.

27. A method for the prevention or treatment of chronic bronchitis, emphysema, asthma, acute lung injury, cystic fibrosis, rhinitis, adult or respiratory distress syndrome (ARDS), urinary incontinence, irritable bowel syndrome, psoriasis, hyperhydrosis, sialorrhea, or a gastrointestinal ulcer, comprising administering to a subject in need thereof an effective amount of diastereoisomerically pure glycopyrronium chloride and/or glycopyrronium chloride prepared according to a method according to claim 1.

28. A method for the prevention or treatment of chronic bronchitis, emphysema, asthma, acute lung injury, cystic fibrosis, rhinitis, adult or respiratory distress syndrome (ARDS), urinary incontinence, irritable bowel syndrome, psoriasis, hyperhydrosis, sialorrhea, or a gastrointestinal ulcer, comprising administering to a subject in need thereof an effective amount of diastereoisomerically pure glycopyrronium chloride and/or glycopyrronium chloride prepared according to a method according to claim 7.

29. A method for the prevention or treatment of chronic bronchitis, emphysema, asthma, acute lung injury, cystic fibrosis, rhinitis, adult or respiratory distress syndrome (ARDS), urinary incontinence, irritable bowel syndrome, psoriasis, hyperhydrosis, sialorrhea, or a gastrointestinal ulcer, comprising administering to a subject in need thereof an effective amount of diastereoisomerically pure glycopyrronium chloride and/or glycopyrronium chloride prepared according to a method according to claim 11.

30. A method for the prevention or treatment of chronic bronchitis, emphysema, asthma, acute lung injury, cystic fibrosis, rhinitis, adult or respiratory distress syndrome (ARDS), urinary incontinence, irritable bowel syndrome, psoriasis, hyperhydrosis, sialorrhea, or a gastrointestinal ulcer, comprising administering to a subject in need thereof an effective amount of diastereoisomerically pure glycopyrronium chloride and/or glycopyrronium chloride prepared according to a method according to claim 16.
